(19) United States
(12) Patent Application Publication
(10) Pub. No.: US 2024/0082364 A1
(43) Pub. Date: Mar. 14, 2024

(54) COMPOSITIONS FOR SUPPLEMENTS AND RELATED METHODS

(71) Applicant: doTERRA Holdings, LLC, Pleasant Grove, UT (US)

(72) Inventors: Alex McGregor DaBell, Springville, UT (US); Nicole Stevens, Pleasant Grove, UT (US)

(21) Appl. No.: 18/463,879

(22) Filed: Sep. 8, 2023

Related U.S. Application Data
(60) Provisional application No. 63/405,281, filed on Sep. 9, 2022.

(51) Int. Cl. A61K 38/39; A61K 8/65; A61K 8/9789; A61K 8/9794; A61K 31/05; A61K 31/132; A61K 31/164; A61K 31/205; A61K 31/23; A61K 31/375; A61K 31/4188; A61K 31/4375; A61K 31/706; A61K 31/7084; A61K 31/728; A61K 33/06; A61K 35/60; A61K 36/185; A61K 36/258; A61K 36/31; A61K 36/48; A61K 36/53; A61K 36/534; A61K 36/605; A61K 36/61; A61K 36/67; A61Q 19/00

(52) U.S. Cl. CPC A61K 38/39; A61K 8/65; A61K 8/9789; A61K 8/9794; A61K 31/05; A61K 31/132; A61K 31/164; A61K 31/205; A61K 31/23; A61K 31/375; A61K 31/4188; A61K 31/4375; A61K 31/706; A61K 31/7084; A61K 31/728; A61K 33/06; A61K 35/60; A61K 36/185; A61K 36/258; A61K 36/31; A61K 36/48; A61K 36/53; A61K 36/534; A61K 36/605; A61K 36/61; A61K 36/67; A61Q 19/00; A61K 2236/331; A61K 2236/333; A61K 2236/37; A61K 2236/51

(57) ABSTRACT

Compositions may include at least one whole or partial CPTG essential oil component, at least one marine or other collagen type component including greater than 70% tripeptides, and at least one whole or partial constituent component. Methods for preparing essential oil food product formulations for animal or human consumption may involve mixing at least one whole or partial CPTG essential oil component, at least one marine or other collagen type component comprising greater than 70% tripeptides, at least one whole or partial constituent component, and a liquid until at least substantially homogenous. Other compositions may include at least one essential oil component and at least one marine or other collagen type component. A ratio by weight of the at least one essential oil component to the at least one marine or other collagen type component may be within a range of from about 2:0 to about 10:1.

### CROSS-REFERENCE TO RELATED APPLICATION

This application claims the benefit under 35 U.S.C. § 119(e) of U.S. Provisional Patent Application Ser. No. 63/405,281, filed Sep. 9, 2022, for COMPOSITIONS FOR SUPPLEMENTS AND RELATED METHODS, the disclosure of which is incorporated herein in its entirety by this reference.

### FIELD

This disclosure relates generally to the field of nutrition and in particular food product formulations based on combining dōTERRA® Certified Pure Tested Grade Essential Oils with marine or other collagen tripeptides plus additional constituents for animal or human consumption, and methods and uses thereof. More specifically, disclosed embodiments relate to novel compositions, which may be marketed as dōTERRA MetaPWR™, and which may include ingestible essential oils in disclosed ratios comprised and combined with disclosed amounts and ratio(s) of marine collagen tripeptides, and natural constituents. When combined, resulting compositions may provide positive outcomes for metabolic function and health, as well as other benefits.

### BACKGROUND

Topical and ingestible supplements for humans and animals may be utilized to enhance health and wellness.

### BRIEF SUMMARY

In some examples, compositions may include at least one whole or partial CPTG essential oil component, at least one marine or other collagen type component including greater than 70% tripeptides, and at least one whole or partial constituent component.

In other examples, methods for preparing essential oil food product formulations for animal or human consumption may involve mixing at least one whole or partial CPTG essential oil component, at least one marine or other collagen type component comprising greater than 70% tripeptides, at least one whole or partial constituent component, and a liquid until at least substantially homogenous.

In other examples, compositions may include at least one essential oil component and at least one marine or other collagen type component. A ratio by weight of the at least one essential oil component to the at least one marine or other collagen type component may be within a range of from about 2:0 to about 10:1.

In accordance with embodiments of this disclosure, methods of delivery for compositions of Certified Pure Tested Grade Essential Oils combined Marine Collagen Tri-peptides and constituents such as vitamins, minerals, phytonutrients, developed to help deliver to healthy human and animals, and those with metabolic challenges, hair, skin and nail health, those with compromised blood glucose levels, lower collagen and NAD+ levels, and those with pre-diabetic conditions with a unique system composed of composition(s) and deliveries as outlined below.

In one aspect, disclosed are compositions comprising Certified Pure Tested Grade Essential Oils from Grapefruit (Citrus paradisi) peel, Lemon (Citrus limon) peel, Peppermint (Mentha piperita) plan, Ginger (Zingiber officinale) root, Cinnamon (Cinnamomum zeylanicum) bark, combined with Mulberry Leaf extract, Berberine HCl, Cinnamon (Cinnamomum loureirii) bark powder, plus Marine Collagen (70% Tripeptides), natural or synthetically derived NMN (nicotinamide mononucleotide) from yeast or other natural constituent, NADH (nicotinamide adenine dinucleotide), Liposomal Vitamin C, Resveratrol, Sea Buckthorn, Rose Apple extract, Phytoceramides from rice or wheat, Hyaluronic Acid (wherein both the at least one whole or partial ingestible component. The CPTG (Certified Pure Tested Grade) essential oil whole or partial ingestible component and one whole or partial collagen source and one whole or partial constituent(s) component can be consumed daily to help promote healthy metabolic function.

In another aspect, the composition can comprise at least 5 separate and/or whole or partial essential oil components, or, in further aspects, at least 12 separate whole or partial constituents or phytonutrient components. The method for distillation, can use steam distillation, solvent extraction, cold pressing, microwave extraction, recirculating method, super critical fluid extraction carbon dioxide extraction CO₂, and/or aqueous extraction, and other future novel and innovative methods. The method for constituent delivery may use embodiments achieved using rapid zone drying, high pressure, window refractance, vacuum belt drying, or other low-temperature drying methods and/or any future constituent delivery methods.

In another aspect, essential oils and/or phytonutrients can, in further aspects, be present in the amount from 0.1 mcg to 50,000 mg/day (1000 ng to 50 g/day) from each source. Phytonutrients from essential oil components include, but not limited to, limonene, gingerols, zingerberine, alpha-pinene,

In another aspect, Constituent compounds may include in the amount of 0.1 mcg to 50,000 mg/day from each source: chlorophyll, lutein, glutathione, alpha lipoic acid, D-glucaric acid, saponins, patuletin, spinacetin, spinatoside, rutin, or lignans. Phytonutrients from red pigment group components include, but not limited to, lycopene, zeta carotene, kaempferol, quercetin, p-coumaric acid, delphyinidin, malvidin, cyanidin, chlorogenic acid, phytoene, phytofluene, epigallocatechin gallate, ellagic acid, benzoic acid, lignans, myricetin, ferulic acid. Phytonutrients from yellow/orange pigment group components include, but are not limited to, hesperetin, limonin, nomilin, d-limonene, beta-sitosterol, mangiferine, syringic acid, beta cryptoxanthin, ferulic acid, chlorogenic acid, or vanillic acid. Additional phytonutrients from essential oil components include, but not limited to, cyaniding, delphinidin, malvidin, peonidin, petunidin, catechins, geraniol, resveratrol, tartaric acid, quercitin, tannins, saponins, caffeic acid, chloregenic acid, sorbitol, or malic acid.

In another aspect, the composition can have a decreased likelihood of stability issues in a sealed package rather than a single-component composition. The compositions can be used for animal or human consumption in a smoothie, fruit drink, shake, food coloring, flavoring, baby food, sports supplemental product, or other food product, homeopathic medicines, oral rinses, oral treatments, medical foods, medical devices, over-the-counter medicines, complementary and alternative therapies, prescription medications, prophylactic and preventive therapies. In another aspect, the composition can be used in a product for topical use that is applied to body surfaces (e.g., skin or mucous membrane) or hair, such as, but not limited to, a lotion, cream, makeup, soap, gel, foam, ear drops, eye drops, inhalers (e.g., dry powder inhalers, metered-dose inhalers, nebulizers, diffusers, aromatic inhalers, vaporizers, humidifiers), mist and ointment. In another aspect, the composition can be used in a product for sun protection, including but not limited to sunscreen lotion or spray. In still other examples, the compositions can further comprise additional ingredients such as herbs, spices, vitamins, minerals, food additives for preservation of and/or improved taste, freshness, or mouthfeel, or a combination thereof.

In a further aspect, the one or more Certified Pure Tested Grade essential oil(s) combined with any of the constituents listed may each have a 1-99 weight percent of the composition.

In a further aspect, the composition may be formulated as a liquid oil, emulsion, effervescent tablet, gum, beadlet, softgel, powder, flakes, tablet, dissolvable film, liquid spray, dropper, caplet, or capsule.

In another aspect, use of the composition as a nutritional source or supplement for humans.

In another aspect, a method for preparing essential oil plus collagen plus constituent formulation for animal or human consumption comprising blending the composition and a liquid (e.g., water) in a shaker cup, ready to drink, squeezable liquid concentrate, stick pack, pouch, sachet, blender and/or added to water or a favorite beverage and then consumed.

### DETAILED DESCRIPTION

Throughout this application, several publications (including, without limitation, patents) are referenced herein. The disclosures of these publications in their entireties are hereby incorporated by reference in this application.

As used herein, the terms “substantially” and “about” in reference to a given parameter, property, or condition means and includes to a degree that one of ordinary skill in the art would understand that the given parameter, property, or condition is met with a degree of variance, such as within acceptable manufacturing tolerances. For example, a parameter that is substantially or about a specified value may be at least about 90% the specified value, at least about 95% the specified value, at least about 99% the specified value, or even at least about 99.9% the specified value.

The illustrative embodiments herein described in the detailed description are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and made part of this disclosure.

The vast majority of adult Americans have poor metabolic health and function and are overweight, obese and have compromised blood glucose levels, and biological excess amounts of adipose fat cells according to the Centers for Disease Control. (National Health Statistics Reports, NHSR No. 158, Jun. 14, 2021) A growing and large percentage of those unknowingly have pre-diabetes or have been diagnosed with diabetes and/or metabolic syndrome. Consuming healthy calorie levels and daily exercise have been shown to mitigate and prevent disease and prolong health-span. The guidelines contained in the Centers for Disease Control booklet titled “Strategies to Prevent Obesity and Other Chronic Diseases” recommends five servings daily of fruits and vegetables. However, over 90% of American adults eat less than 1 fruit or vegetable a day. (Disparities in State Specific Adult Fruit and Vegetable Consumption United States 2015 Weekly, Nov. 17, 2017/66(45); 1241-1247.) To achieve improved health, plant foods should comprise 80% of dietary intake. Specific ingestible essential oils as well as consuming fruits, vegetables, roots and stems and herbal botanicals and phytonutrient constituents have also demonstrated normalized blood glucose levels, improved body composition, increase in NAD+, collagen synthesis, cognitive function, epidermal appearance/health and adipogenesis fat health benefits. (Higher intake of fruits, vegetables or their fiber reduces the risk of type 2 diabetes: A meta-analysis J. Diabetes Investig. 2016 January; 7(1):56-69.) Additionally, the components associated with essential oils have a specific purpose for the animal or human body. Each essential oil includes phytochemicals and/or which each have its own mechanism of action and function.

The World Health Organization (WHO) recommends that all adults participate in 150-300 minutes of moderate intense aerobic activity, and 75-150 minutes of vigorous intense physical activity throughout the week. Additionally, WHO recommends limiting the amount of time spent being sedentary and replacing sedentary time with physical activity of any intensity to help reduce the detrimental effects of metabolic syndrome. Physical activity is essential to promote healthy metabolic health and function.

Upon reaching adulthood, the number of fat cells in the human body stays the same. These adipose fat cells can grow and shrink and 5-10% die each year and are then replaced. As adults age, physiologically the body's metabolism begins to slow down. Energy that was once created efficiently and effectively becomes harder to convert to adenosine triphosphate (ATP). (Nature 2008 Hopkin, M. Fat cell numbers stay constant through adult life. 5 May 2008 https://doi.org/10/1038/news.2008.800).

Preclinical research suggests that grapefruit essential oil may decrease food consumption and subsequently, body weight. In addition, this research suggests that it may promote healthy metabolism of fat. J. Shen, A. Niijima, M. Tanida, Y. Horii, K. Maeda, and K. Nagai, “Olfactory stimulation with scent of grapefruit oil affects autonomic nerves, lipolysis and appetite in rats,” Neurosci. Lett., vol. 380, no. 3, pp. 289-294, June 2005, doi: 10.1016/j.neulet.2005.01.058. J. Shen, A. Niijima, M. Tanida, Y. Horii, T. Nakamura, and K. Nagai, “Mechanism of changes induced in plasma glycerol by scent stimulation with grapefruit and lavender essential oils,” Neurosci. Lett., vol. 416, no. 3, pp. 241-246, April 2007, doi: 10.1016/j.neulet.2006.12.063. A. Niijima and K. Nagai, “Effect of olfactory stimulation with flavor of grapefruit oil and lemon oil on the activity of sympathetic branch in the white adipose tissue of the epididymis,” Exp. Biol. Med. Maywood NJ, vol. 228, no. 10, pp. 1190-1192, November 2003, doi: 10.1177/153537020322801014.

The monoterpene limonene is the main chemical constituent in grapefruit and lemon essential oil. Preclinical research suggests that high amounts of orally administered limonene may protect against negative effects associated with a high fat diet and promote healthy levels of sugars in the blood. J. Victor Antony Santiago, J. Jayachitra, M. Shenbagam, and N. Nalini, “Dietary d-limonene alleviates insulin resistance and oxidative stress-induced liver injury in high-fat diet and L-NAME-treated rats,” Eur. J. Nutr., vol. 51, no. 1, pp. 57-68, February 2012, doi: 10.1007/s00394-011-0182-7.

Preclinical research suggests that oral administration of limonene may promote the breakdown of fats and healthy levels of sugar in the blood caused by excess body weight and a high fat diet. Laboratory research supports these results as tested in fat cells. L. Jing et al., “Preventive and ameliorating effects of citrus D-limonene on dyslipidemia and hyperglycemia in mice with high-fat diet-induced obesity,” Eur. J. Pharmacol., vol. 715, no. 1-3, pp. 46-55, September 2013, doi: 10.1016/j.ejphar.2013.06.022.[6]X. C. Tan, K. H. Chua, M. Ravishankar Ram, and U. R. Kuppusamy, “Monoterpenes: Novel insights into their biological effects and roles on glucose uptake and lipidmetabolism in 3T3-L1 adipocytes,” Food Chem., vol. 196, pp. 242-250, April 2016, doi: 10.1016/j.foodchem.2015.09.042. Vieira, “Limonene: Aroma of innovation in health & disease-ScienceDirect.” https://www.sciencedirect.com/science/article/pii/S0009279717310487?via %3Dihub (accessed Mar. 12, 2020).

Clinical research suggests that chronic consumption of larger amounts of limonene may ensure that limonene reaches the tissues needed to have an effect (fat and blood). Preclinical studies suggest that oral administration of peppermint oil may promote healthy digestion of fat in the gut. Laboratory research suggests that grapefruit essential oil may prevent fat accumulation (inhibits adipogenesis). Thus, the dōTERRA MetaPWR Oil Blend may promote healthy metabolism of fats. See J. A. Miller, I. A. Hakim, W. Chew, P. Thompson, C. A. Thomson, and H.-H. S. Chow, “Adipose tissue accumulation of d-limonene with the consumption of a lemonade preparation rich in d-limonene content,” Nutr. Cancer, vol. 62, no. 6, pp. 783-788, 2010, doi: 10.1080/01635581003693066. L. Zong et al., “Preliminary experimental research on the mechanism of liver bile secretion stimulated by peppermint oil,” J. Dig. Dis., vol. 12, no. 4, pp. 295-301, 2011, doi: 10.1111/j.1751-2980.2011.00513.x. S. Haze, K. Sakai, Y. Gozu, and M. Moriyama, “Grapefruit oil attenuates adipogenesis in cultured subcutaneous adipocytes,” Planta Med., vol. 76, no. 10, pp. 950-955, July 2010, doi: 10.1055/s-0029-1240870. T. T. Le, “(MetaPWR) Report_220523 (adipose cells),” May 12, 2021 (unpublished).

Clinical research suggests that ingestion of peppermint oil may help to decrease appetite. A. Papathanasopoulos et al., “Effect of acute peppermint oil administration on gastric sensorimotor function and nutrient tolerance in health,” Neurogastroenterol. Motil. Off. J. Eur. Gastrointest. Motil. Soc., vol. 25, no. 4, pp. e263-271, April 2013, doi: 10.1111/nmo.12102.

Clinical research suggests that peppermint oil supplementation may improve cardiovascular and respiratory parameters during exercise. A. Meamarbashi, A. Rajabi, and others, “The effects of peppermint on exercise performance,” J Int Soc Sports Nutr, vol. 10, no. 1, p. 15, 2013.

Ginger oil has been demonstrated to help increase energy and stomach calming benefits. A descriptive clinical study (n=87) in children showed that inhalation of ginger essential oil made males feel energetic, however it was least likely, along with lavender oil, to be selected by subjects to take home. Method of evaluation was a questionnaire with 3 forced choice questions and one open ended question. M. Fitzgerald, T. Culbert, M. Finkelstein, M. Green, A. Johnson, and S. Chen, “The Effect of Gender and Ethnicity on Children's Attitudes and Preferences for Essential Oils: A Pilot Study,” EXPLORE J. Sci. Heal., vol. 3, no. 4, pp. 378-385, July 2007, doi: 10.1016/j.explore.2007.04.009. A single-blind, randomized, placebo-controlled, crossover clinical study (n=60) in women showed that inhalation of ginger essential oil (2 drops on aromatherapy necklace for 5 days) significantly increased energy intake in breast-cancer patients experiencing chemotherapy-induced nausea and vomiting. This was measured by physiological parameters (BMI) and food records. N. Salihah, N. Mazlan, and P. L. Lua, “The effectiveness of inhaled ginger essential oil in improving dietary intake in breast-cancer patients experiencing chemotherapy-induced nausea and vomiting,” Focus Altern. Complement. Ther., vol. 21, no. 1, pp. 8-16, 2016, doi: 10.1111/fct.12236.

Clinical research shows that collagen supplementation promotes healthy bones and joints. Bone and joint pain or issues are commonly associated with aging [26]-[29]. Preclinical research supports these findings. A. Czajka et al., “Daily oral supplementation with collagen peptides combined with vitamins and other bioactive compounds improves skin elasticity and has a beneficial effect on joint and general wellbeing,” Nutr. Res., vol. 57, pp. 97-108, September 2018, doi: 10.1016/j.nutres.2018.06.001. P. Benito-Ruiz et al., “A randomized controlled trial on the efficacy and safety of a food ingredient, collagen hydrolysate, for improving joint comfort,” Int. J. Food Sci. Nutr., vol. 60, no. sup2, pp. 99-113, January 2009, doi: 10.1080/09637480802498820. K. L. Clark et al., “24-Week study on the use of collagen hydrolysate as a dietary supplement in athletes with activity-related joint pain,” Curr. Med. Res. Opin., vol. 24, no. 5, pp. 1485-1496, May 2008, doi: 10.1185/030079908X291967. P. Dressler, D. Gehring, D. Zdzieblik, S. Oesser, A. Gollhofer, and D. König, “Improvement of Functional Ankle Properties Following Supplementation with Specific Collagen Peptides in Athletes with Chronic Ankle Instability,” J. Sports Sci. Med., vol. 17, no. 2, pp. 298-304, May 2018. S. Takeda, J.-H. Park, E. Kawashima, I. Ezawa, and N. Omi, “Hydrolyzed collagen intake increases bone mass of growing rats trained with running exercise,” J. Int. Soc. Sports Nutr., vol. 10, no. 1, p. 35, August 2013, doi: 10.1186/1550-2783-10-35.

In review of clinical and preclinical research, supplementation of amino acids common in marine collagen (glycine, proline, hydroxyproline, and lysine) helped promote metabolic health and increased longevity due to enhancing a healthy cardiovascular system, circulatory system, nervous system, metabolism of sugars and fats, and weight management. The quality of amino acids plays a role in the beneficial impacts. Restriction of certain amino acids helps promote longevity by improving metabolic health through promoting healthy functioning of the cardiovascular and circulatory system, metabolism of blood sugars, and nervous system. Activation of the AMPK pathway occurred. The marine collagen may give the benefits of quality amino acids without including amino acids that can cause harm. In a clinical study, supplementation of collagen tripeptides promoted a healthy cardiovascular system. Preclinical research supports these findings. P. Berillis, “Marine Collagen: Extraction and Applications,” p. 14. G. Wu, “Amino acids: metabolism, functions, and nutrition,” Amino Acids, vol. 37, no. 1, pp. 1-17, May 2009, doi: 10.1007/s00726-009-0269-0. [33]R. Babygirija and D. W. Lamming, “The regulation of healthspan and lifespan by dietary amino acids,” Transl. Med. Aging, vol. 5, pp. 17-30, 2021, doi: 10.1016/j.tma.2021.05.001. G. Wu, “Functional amino acids in nutrition and health,” Amino Acids, vol. 45, no. 3, pp. 407-411, September 2013, doi: 10.1007/s00726-013-1500-6. C. L. Green and D. W. Lamming, “Regulation of metabolic health by essential dietary amino acids,” Mech. Ageing Dev., vol. 177, pp. 186-200, January 2019, doi: 10.1016/j.mad.2018.07.004. N. Tomosugi et al., “Effect of Collagen Tripeptide on Atherosclerosis in Healthy Humans,” J. Atheroscler. Thromb., vol. advpub, p. 36293, 2016, doi: 10.5551/jat.36293. L. Tang, Y. Sakai, Y. Ueda, and S. Katsuda, “Effects of oral administration of tripeptides derived from type I collagen (collagen tripeptide) on atherosclerosis development in hypercholesterolemic rabbits,” J. Biosci. Bioeng., vol. 119, no. 5, pp. 558-563, May 2015, doi: 10.1016/j.jbiosc.2014.10.011.

In a randomized, double-blind, placebo-controlled clinical study (n=108), supplementation with NMN (250 mg for 12 weeks) improved physical performance and reduced fatigue as determined by Pittsburgh Sleep Quality Index, a questionnaire, and physical performance tests (grip strength, 5-times sit-to-stand, timed up and go, and 5-m habitual walk). In a randomized, double-blind, placebo-controlled, four-arm clinical trial, athletes (n=48) who were supplemented NMN (300, 600, and 1200 mg/day for 6 weeks) showed an increase in aerobic capacity during exercise training as measured by cardiopulmonary exercise testing. M. Vasile, A. Bunea, C. R. Joan, B. C. Joan, S. Socaci, and M. Viorel, “Phytochemical Content and Antioxidant Activity of Malusdomestica Borkh Peel Extracts,” Molecules, vol. 26, no. 24, p. 7636, December 2021, doi: 10.3390/molecules26247636. M. Kim, J. Seol, T. Sato, Y. Fukamizu, T. Sakurai, and T. Okura, “Effect of 12-Week Intake of Nicotinamide Mononucleotide on Sleep Quality, Fatigue, and Physical Performance in Older Japanese Adults: A Randomized, Double-Blind Placebo-Controlled Study,” Nutrients, vol. 14, no. 4, Art. no. 4, January 2022, doi: 10.3390/nu14040755. B. Liao, Y. Zhao, D. Wang, X. Zhang, X. Hao, and M. Hu, “Nicotinamide mononucleotide supplementation enhances aerobic capacity in amateur runners: a randomized, double-blind study,” J. Int. Soc. Sports Nutr., vol. 18, no. 1, p. 54, January 2021, doi: 10.1186/s12970-021-00442-4.

In a randomized, controlled clinical trial involving subjects with BMI≥30 kg/m2(n=25), oral supplementation with resveratrol (250 mg/day for 3 months) in combination with diet and physical activity significantly increased high-density lipoprotein cholesterol and reduced total cholesterol and very-low density lipoprotein cholesterol in the serum compared to placebo treatment plus diet and physical activity as determined by nutritional assessments, anthropometric measurements, and biochemical measurements. G. C. Batista-Jorge et al., “Oral resveratrol supplementation improves Metabolic Syndrome features in obese patients submitted to a lifestyle-changing program,” Life Sci., vol. 256, p. 117962, September 2020, doi: 10.1016/j.lfs.2020.117962.

Vitamin C is a water-soluble vitamin and is a dietary antioxidant that promotes the regeneration of other antioxidants in the body. A systematic review on cerebral health and aging suggested that supplementation of vitamin C may be effective in combating oxidative stress. European Food Safety Authority (EFSA) has opined that supplementation of vitamin C contributes to the collagen formation for skin, bone, and cartilage functioning and maintenance. Vitamin C plays a number of physiological roles in maintaining skin structure and function; high levels of vitamin C are found in the dermis and epidermis; vitamin C limits skin damage by preventing UV damage, oxidative stress, and promoting collagen synthesis. In a review of randomized, controlled, prospective, cross-sectional, and case-control clinical studies, supplementation of vitamin C showed a potential association with cognition. In a review, vitamin C supplementation showed an effect on the nervous system by improving neurotransmission which enhanced memory, learning, and locomotion. X. Capó, M. Martorell, A. Sureda, J. A. Tur, and A. Pons, “Effects of Docosahexaenoic Supplementation and In Vitro Vitamin C on the Oxidative and Inflammatory Neutrophil Response to Activation,” Oxid. Med. Cell. Longev., vol. 2015, 2015, doi: 10.1155/2015/187849. H. R. Freitas, G. da C. Ferreira, I. H. Trevenzoli, K. de J. Oliveira, and R. A. de Melo Reis, “Fatty Acids, Antioxidants and Physical Activity in Brain Aging,” Nutrients, vol. 9, no. 11, November 2017, doi: 10.3390/nu9111263. “European Food Safety Authority (EFSA)-Committed to ensuring that Europe's food is safe.” http://www.efsa.europa.eu/ (accessed Jan. 30, 2015). “Office of Dietary Supplements—Vitamin C.” https://ods.od.nih.gov/factsheets/VitaminC-HealthProfessional/ (accessed Mar. 9, 2020). N. Travica, K. Ried, A. Sali, A. Scholey, I. Hudson, and A. Pipingas, “Vitamin C Status and Cognitive Function: A Systematic Review,” Nutrients, vol. 9, no. 9, p. 960, August 2017, doi: 10.3390/nu9090960. G. Grosso et al., “Effects of Vitamin C on health: a review of evidence,” Front. Biosci.-Landmark, vol. 18, no. 3, Art. no. 3, June 2013, doi: 10.2741/4160.

Daily consumption of fruits and vegetables has also been shown to mitigate or ameliorate the risks of more serious health conditions. For example, in a recent prospective cohort study of 41,837 postmenopausal women, the association of fruit and vegetable consumption with lung cancer risk was investigated. Researchers found that the risk of lung cancer was cut in half when the consumption of fruits and vegetables increased from 24 or less servings to an excess of 48 servings per week. Similarly, the risk of lung cancer was also cut in half when the consumption of green leafy vegetables, including spinach and parsley sources, increased from 1 or fewer servings to 6 or more servings per week. Steinmetz, K. et al., “Vegetables, Fruit, and Lung Cancer in the Iowa Women's Health Study,” Cancer Res. 53:536-43 (1993). Another study found that an increased intake of fresh tomatoes (a major source of lycopene) was associated with a pattern of protection for all sites of digestive tract cancer. Stahl, W. et al., “Lycopene: A Biologically Important Carotenoid for Humans?” Arc. Biochem. Biophys. 336:1-9 (1996).

Additionally, studies have also shown that diets high in plant foods are associated with reduced risk of various types of cancers. These studies correlate diets rich in fruits and vegetables and nutrients from these diets, such as vitamin C, vitamin A, and dietary fiber, with reduced cancer risk. Persons consuming these diets frequently have high intakes of these nutrients.

There are many barriers that prevent consumption of essential oils, collagen, whole foods, plants and nutrient rich sources. Geographically, there are areas deprived of nutrient-rich food sources where people live who are not adequately nourished or do not have access to ocean, streams, fresh water lakes, fruits, herbs and phytonutrients that are stable and still retain their potency and nutrient density. Further, most essential oils are volatile, fish can spoil quickly and fruits and vegetables lose nutrients quickly because they are susceptible to mold, bacteria and fungus which can grow rapidly. Finally, many households do not use up the ingestible fruits and vegetables they buy. Once picked or prepared, many consumers are unable to use up the entirety of a fruit or vegetable because of the short shelf life of the produce.

There remains a need, therefore, for convenient vehicles for certified essential oils, combined with phytonutrients, herbs, vitamins and other essential nutrients, which have potent essential nutrients, pigments and phytonutrients high in biomedically active plant nutrients, but that are stable and have a lengthy shelf-life.

In accordance with embodiments disclosed herein, Certified Pure Tested Grade essential oils plus ingestible constituents' compositions may provide a stable source of metabolically active essential oils, collagen sources, constituents, compounds and phytonutrients with a long shelf-life, and health benefits not decreased from the fresh forms of plants, stems, oils, the fruits and vegetables. Also, such compositions may be formulated in a manner to promote convenience of use and improved cellular absorption. Further, the composition may be at least substantially free of artificial flavors, preservatives, colors, binders, and/or fillers, outside of normal manufacturing practices, since some preparation process discussed herein (e.g., harvesting, distillation and drying techniques) may preserve phytonutrients, pigments, and other health-enriching characteristics of each nutrient rich oil, tripeptide collagen sources, and constituents used in the compositions discussed herein.

Certified Pure Tested Grade (CPTG) Essential Oils

CPTG Essential oils may be harvested and distilled at or near the time of peak richness, freshness, aroma, taste, appearance, condition, and phytoactive value. Because of the volatile nature of essential oils, increasing harvesting and handling speed can preserve and increase quality. Any techniques known to those in the art may be used to extend postharvest shelf life, including potential removal from direct sunlight, and reducing the respiration by hydro or rapid cooling; slow respiration by maintaining controlled shipping and storing environment; or maintaining controlled relative temperature and humidity. Special skills may be used for proper harvesting, handling, grading to ensure optimum quality. Essential oil leaves, roots, bark, seeds, fruit, flower, stems, peels may be prepared, utilizing care and in accordance with quality requirements. Additional measures are taken to ensure high quality, premium, Certified Pure Tested Grade (CPTG) pure essential oils including optionally performing eight quality tests including 1. Organoleptic testing, 2. Microbial testing, 3. Gas chromatography, 4. Mass spectrometry, 5. Fourier-transform infrared spectroscopy (FTIR), 6. Chirality testing, 7. Isotopic analysis, 8. Heavy metal testing and up to 140+ testing methods depending on the essential oil source and type.

The Certified Pure Tested Grade (CPTG) essential oils and constituent compositions described herein are grouped by efficacy and efficiency in formulation. Generally, essential oils can be grouped together by liquid delivery when in oil form and when dried combined with dried constituents.

Each essential oil with its constituent component can be provided in any concentration. For example, each component can be about 1% by weight, about 2% by weight, about 4% by weight, about 5% by weight, about 10% by weight, about 15% by weight, about 18% by weight, about 20% by weight, about 22% by weight, about 23% by weight, about 24% by weight, about 25% by weight, about 30% by weight, about 35% by weight, about 40% by weight, about 45% by weight, about 50% by weight, about 55% by weight, about 60% by weight, about 70% by weight, about 80% by weight, about 90% by weight, or about 99% by weight. Also, each component can have a range (by weight) of 1%-2%; 2%-4%; 1%-5%; 2%-4%; 2%-5%; 4%-6%; 5%-7%; 6%-8%; 7%-10%; 8%-11%; 8%-14%; 9%-14%; 12%-14%; 15%-19%; 15%-22%; 15%-24%; 19%-21%; 19%-24%; 21%-24%; 24%-33%; 25%-30%; 1%-15%; 1%-18%; 1%-20%; 1%-22%; 1%-23%; 1%-24%; 1%-25%; 1%-30%; or 30%-99%.

In some examples, compositions in accordance with this disclosure may include one or more whole or partial Certified Professional Therapeutic Grade (CPTG) essential oil component(s). For example, essential oil compositions may include five or more whole or partial essential oil components and/or twelve or more whole or partial constituent components. Such essential oil components and constituent components may provide active compounds and phytonutrients in certain ratios and levels present in a portion ranges of approximately 0.1 mcg to 50,000 mg/day or (1000 mcg to 50 g/day) from a terpenes and limonene sources including but not limited grapefruit peel, lemon peel, orange peel, lime peel, bergamot peel, fruit extract, blood orange peel, yuzu peel, mandarin peel, tangerine peel and all other citrus peel varieties; which provide phytonutrients in certain ratios and levels present in a portion ranges of approximately 0.1 mcg to 50,000 mg/day. Cinnamon sources include but not limited to cinnamaldehyde, Cinnamomum verum (True cinnamon), C. cassia (cassia cinnamon), C. citriodorum, C. loureiroi (Saigon cinnamon), C. burnmanni, C. zeylanicum, (Ceylon cinnamon), which provide phytonutrients in certain ratios and levels present in a portion ranges of approximately 0.1 mcg to 50,000 mg/day. Peppermint (Mentha piperita) sources include but not limited to more than 25+ mint species, e.g., bergamot mints, spearmint, which provide phytonutrients menthol, menthone, iso-menthone, linalool, linalyl acetate, carvone, limonene, pulegone, caryophyllene, pinene, terpenoids, sesquiterpinoids in certain ratios and levels present in ranges of approximately 0.1 mcg to 50,000 mg/day.

Some compositions described herein may include ratios and levels of enzymes, organic substances, metabolites, phytonutrient families, terpenes, carotenoids, polyphenolics, anythocyanins, glycosides, polysaccharides, flavonoids, from various whole food sources including but not limited to: rasaceae, vitaceae, rutaceae, lythraceae, schisandraceae, cucrbitaceae, sapindaceae, rhammnaceae, malvaceae, elaeagnaceae, formulated by essential oil plus collagen types, plus constituents by efficacy and effectiveness from 5 or more essential oils, plus collagen tripeptides plus additional constituents. Such substances in these compositions have been shown to aid in the inhibition of fat cell maturation and creation of new fat cells, (steatosis and adipogenesis), normalization of blood glucose, insulin response, NAD+ production, collagen degradation, cognitive function, skin health, and metabolic performance. Combined with healthy lifestyle choices the prevention and/or treatment of tumor growth, cancer formation, cancer diffusion, abnormal angiogenesis, neurodegenerative disorder, diabetes, inflammation, atherosclerosis, viral infection, toxicity, glycemia, oxidation, leishmanial, choleretic, spasmodic, immune and autoimmune disease, blood coagulation, osteoarthritis, rheumatoid arthritis, cataracts, liver malfunction, kidney disease, blood sugar disorders, fatigue, wound healing, and metabolic syndrome.

Essential Oil Distillation, Constituent and Phytonutrient Drying/Dehydration Techniques

The one or more whole or partial CPTG essential oils components for compositions described herein can be derived using, for example, steam distillation, gentle pressure, cold pressing, solvent expression with hydrosol or CO2, condensing, and cooling. Drying methods using any low-temperature drying mechanism known in the art. For example, the compositions can be dried using rapid zone drying, window refractance, vacuum belt drying, or other low temperature drying. For example, the method of U.S. Pat. No. 4,631,837 can be used, which involves floating an infrared-transparent on the surface of a body of heated water and placing the material to be dried, such as the pulp and herbal or vitamin mix, on top of the film to absorb heat from the water and thereby cause drying/dehydration of the material. The apparatus for carrying out this drying method includes a reservoir of water, a film floated on the water, and a heater for maintaining the temperature of the water at a predetermined level. In some examples, the reservoir may be in the form of an elongated trough and the film comprises an endless strip of polyester material, such as Mylar, to form a conveyor belt that floats on the surface of the water in the trough and carries the material to be dried on it.

In another example, the method of U.S. Pat. No. 6,539,645 can be used for drying/dehydrating of the compositions. The method involves placing the product on a first side of a support surface and directing dry infrared light toward the second side of the surface to remove moisture from the product. A sensor can be included to measure at least one characteristic of the product, such as the temperature or moisture content thereof. The temperature of the light source can be regulated as a function of the measured characteristic. The support surface can also be made to be movable relative to the light source. In an alternative method, a plurality of control zones may be defined and through which the product is successively passed. Each of the control zones has at least one associated light source and an associated sensor to regulate the temperature of the light sources associated with each control zone independently of those associated with another zone.

The compositions described herein can also include any other ingredients deemed desirable or known in the art, such as, but not limited to, the following: vitamins, minerals, antioxidants, spices, herbs, extracts, phytonutrients, and/or other food additives for the preservation of taste, freshness, consistency, mouthfeel, or any combination or subcombination thereof. Such additives are well known in the art.

Illustrative Certified Pure Tested Grade Essential Oils from Grapefruit (Citrus paradisi) peel, Lemon (Citrus limon) peel, Peppermint (Mentha piperita) plant, Ginger (Zingiber officinale) root, Cinnamon (Cinnamomum zeylanicum or Cinnamomum cassia) bark, combined with Mulberry Leaf extract, Berberine HCl, Cinnamon (Cinnamomum loureirii) bark powder, plus Marine Collagen (greater than or equal to 70% Tripeptides), natural or synthetically derived NMN (Nicotinamide Mononucleotide) from yeast or other constituent, NADH (Nicotinamide adenine dinucleotide), Liposomal Vitamin C, Resveratrol, Sea Buckthorn, Rose Apple extract, Phytoceramides from rice or wheat, Hyaluronic Acid compositions are described as follows in TABLE 1:

TABLE 1

| | Marine or Other | |
|---|---|---|
| Certified Pure Tested | Collagen with > | |
| Grade (CPTG) Essential | or = 70% Tripeptide | |
| Oil Sources | Sources | Source for Constituent Component(s) |
| Grapefruit peel | Tilapia | Mulberry Leaf |
| Lemon Peel | Cod | Berberine HCl |
| Peppermint | Basa | MCT Oil (medium chain tri-glycerides) |
| Ginger | Sea Bass | NMN Yeast (Nicotinamide Mononucleotide) |
| Cinnamon | Rohu | NADH (Nicotinamide Adenosine Dinucleotide) |
| Orange(s) (all) Peel | Catia | NR (Nicotinamide Riboside) |
| Lime Peel | Saddletail sea perch | Liposomal Vitamin C |
| Mandarin peel | Crimson sea perch | Resveratrol |
| Tangerine peel | Black porgy | Phytoceramides |
| Pineapple peel | Golden threadfin bream | Rose Apple |
| Mango peel | Flat fish | Vitamin B7 (Biotin) |
| Ugli fruit peel | Grass fish | Hyaluronic Acid |
| Kaffir lime peel | Lobotidae | Broccoli Sprout Powder |
| Pummelo peel | Milkfish | Vitamin B3 |
| Rough Lemon peel | Snakehead | Pomegranate |
| Tangelo peel | Pangasius | Spermidine |
| Satsuma mandarin | Pollock | Baobab Extract |
| Kumquat peel | Haddock | Sodium/Potassium 1:3 Ratio |
| Pomelo peel | Beef | Magnesium/Calcium |
| White grapefruit | Pork | Molybdenum |
| Yuzu peel | | OEA (Oleoylethanolamide) |
| Sudachi peel | | Black cumin seed |
| Copaiba | | Thymoquinone |
| Coriander | | Chromium picolinate |
| Cumin Seed | | PQQ (pyrroloquinoline quinone) |
| Lemon Grass | | Yerba Santa (Eriodictyon californicum) |
| Bergamot | | L-Carnitine |
| Melissa (Lemon balm) | | Fresh Ginseng |

Additional embodiments within the scope of this disclosure are presented in greater detail in connection with the following non-limiting examples.

### EXAMPLES

### Example 1—Formulation of CPTG Essential Oils+Marine or Other Collagen Sources+Constituents Compositions

Compositions in accordance with this disclosure may also include one or more marine collagen or other collagen type component(s). For example, some illustrative compositions may include nine or more marine or other collagen type components. To formulate the essential oil+marine or other collagen+constituent compositions, ingestible compounds may be sustainably harvested, distilled and dried at the time of desired nutrient richness, quality, freshness, taste, appearance, condition, and nutritive value. Because of the perishable nature of some sources of plants, fish, and constituents, increasing harvesting and handling quality and speed may maintain and increase the quality of the result. Several techniques that are used to extend postharvest shelf life are known to those of skill in the art, and one or more of the following may be employed: steam or expressed distillation, wild caught or farm harvested, protection from direct sunlight and reducing the respiration by hydro or rapid cooling; slow respiration by maintaining optimal shipping containment and storage environment(s); and/or maintaining optimal relative humidity.

TABLE 2
Essential Oil + Marine Collagen + Constituent COMPOSITIONS

| Component | Latin Binomial or name | Part Used | Weight % |
|---|---|---|---|
| Grapefruit peel | Citrus paradisi | Peel/Skin | <1-90% |
| Lemon Peel | Citrus Limon | Peel/Skin | <1-90% |
| Peppermint | Mentha piperita | Leaf/Stem | <1-75% |
| Ginger | Zingiber officinale | Bark | <1-75% |
| Cinnamon | Cinnamomum zeylanicum | Bark/Oil | <1-75% |
| Orange(s) (all) Peel | Citrus maxima/reticulata | Peel/Skin | <1-90% |
| Lime Peel | Citrus aurantiifolia | Peel/Skin/Rind | <1-90% |
| Mandarin peel | Citrus reticulata blanco | Peel/Skin/Rind | <1-90% |
| Melissa | Melissa officinalis l. | Peel/Skin/Rind | <1-90% |
| Tangerine peel | Citrus tangerina | Peel/Skin/Rind | <1-90% |
| Bergamot peel | Citrus bergamia | Peel/Skin/Rind | <1-90% |
| Ugli fruit peel | Citrus reticulata/paradisi | Peel/Skin/Rind | <1-75% |
| Yuzu peel | Citrus cavaleriei | Peel/Skin/Rind | <1-75% |
| Kaffir lime peel | Citrus hystrix | Peel/Skin/Rind | <1-75% |
| Rough Lemon peel | Citrus jambhiri lush. | Peel/Skin/Rind | <1-75% |
| Tangelo Peel | Citrus tangelo | Peel/Skin/Rind | <1-75% |
| Pummelo Peel | Citrus Maxima | Peel/Skin/Rind | <1-75% |
| Satsuma mandarin | Citrus unshiu | Peel/Skin/Rind | <1-75% |
| Kumquat peel | Citrus japonica/crassifolia | Peel/Skin/Rind | <1-75% |
| White grapefruit | Citrus paradisi | Peel/Skin/Rind | <1-90% |
| Patchouli | Pogostemon Cablin | Stem/Leaf | <1-50% |
| Tilapia | Oreochromis niloticus | Skin/Scale | <1-50% |
| Cod | Theragra chalcogramma | Skin/Scale | <1-50% |
| Basa | Pangasius bocourti | Skin/Scale | <1-50% |
| Sea Bass | Lates calcarifer | Skin/Scale | <1-50% |
| Rohu | Labeo rohita | Skin/Scale | <1-50% |
| Catla | Gibelion catla | Skin/Scale | <1-50% |
| Saddletail sea perch | Lutjanus malabaricus | Skin/Scale | <1-50% |
| Crimson sea perch | Lutjanus erythropterus | Skin/Scale | <1-50% |
| Black porgy | Acanthopagrus schlegeli | Skin/Scale | <1-50% |
| Golden threadfin bream | Nemipterus virgatus | Skin/Scale | <1-50% |
| Flat fish | Pleuronectinae | Skin/Scale | <1-50% |
| Grass fish | Ctenopharyngodon Idella | Skin/Scale | <1-50% |
| Lobotidae | Lobotes pacificus | Skin/Scale | <1-50% |
| Milkfish | Chanos chanos | Skin/Scale | <1-50% |
| Snakehead | Channidae | Skin/Scale | <1-50% |
| Pangasius | Pangasius gigas | Skin/Scale | <1-50% |
| Pollock | G. chalcogrammus | Skin/Scale | <1-50% |
| Haddock | Melanogrammus aeglefinus | Skin/Scale | <1-50% |
| Mulberry Leaf | Morus spp. | Leaf | <1-25% |
| Berberine HCl | B. vulgaris | Bark | <1-25% |
| MCT (medium chain | Medium Chain Triglycerides | Coconut oil | <1-25% |
| triglycerides) | | | |
| NMN | (Nicotinamide Mononucleotide) | Yeast | <1-25% |
| NADH | (Nicotinamide Adenosine Dinucleotide) | Biological form | <1-25% |
| | | Niacin | |
| NR | Nicotinamide riboside | Yeast /Vitamin B3 | <1-25% |
| Liposomal Vitamin C | Ascorbic Acid | Non -GMO corn, | <1-25% |
| | | citrus | |
| Resveratrol | E-5-(4-hydroxystyryl)benzene- | Grape, Yeast, | <1-25% |
| | 1,3-diol | | |
| Phytoceramides | Glycophingolipids | Rice, Konjac Root | <1-25% |
| Rose Apple extract | Syzygium jambos | Rose apple | <1-25% |
| Vitamin B7 | Biotin | Yeast | <1-25% |
| Hyaluronic Acid | Sodium Hyaluronate | Vegetable | <1-25% |
| Broccoli Sprout Powder | Brassica oleracea L. var. italica | Vegetable | <1-25% |
| Pomegranate | Punica granatum L. | Fruit | <1-25% |
| Spermadine | 1,5,10-Triazadecane | Yeast | <1-25% |
| Baobab Fruit Extract | Andansonia digitata L. | Baobab tree | <1-25% |
| Sodium/Potassium 1:3 | Sodium Chloride/Potassium | Salt | <1-25% |
| Ratio | chloride | | |
| Magnesium | (Citrate, Sulphate, L-Threonate, Chloride) | Yeast, vegetable | <1-25% |
| Calcium | Calcium Lactate | Coral, mineral | <1-25% |
| Fresh Ginseng Root | Panax ginseng | Ginseng root | <1-25% |
| Molybdenum | Glycinate, chelate, chloride | Yeast, vegetable | <1-25% |
| Fenugreek | Trigonella foenum-graecum L. | Seed/leaf/stem | <1-25% |
| Oregano | Origanum vulare L. | Leaf/shoots | <1-25% |
| Clove | Syzgium aromaticum L | Leaf/Stem/Flower | <1-25% |
| PQQ | Pyrroloquinoline quinone | Bacteria, bean, | <1-25% |
| | | fruit/vegetable | |
| California Yerba Santa | Eriodictyon californicum | Shrub/leaf | <1-25% |
| Fresh Ginseng | Panax quinquefolius | Root, stem | <1-25% |
| Acetyl l carnitine or L- | Amino acid | Animal/Plant | <1-75% |
| carnitine | | | |

The components and/or weight percentages used in the compositions, such those shown above, can vary based on seasonal availability, growing and environmental conditions, harvest timing, and supply of particular essential oil from fruit, peel, stem, flower, bark, seed, root, leaf or other agriculture-based sources, collagen source and constituent. For example, if a particular component is unavailable due to seasonal frost, another component from the same or another essential oil source can be used as a replacement at the same or different weight percentage.

In some examples, the weight percentage of tripeptides of the marine of other collagen type constituent may be higher than that of other marine or other collagen type constituents known to the inventors. For example, the weight percentage of tripeptides of the marine or other collagen type constituent may be increased or concentrated before the marine or other collagen type constituent is incorporated into the composition. More specifically, the weight percentage of tripeptides of a marine or other collagen type constituent may be increased or concentrated by performing enzymatic hydrolysis on one or more of the marine or other collagen type component(s). As a specific, nonlimiting example, the weight percentage of tripeptides of a marine or other collagen type constituent may be increased or concentrated by performing enzymatic hydrolysis utilizing endo- and exo-peptidase enzymes on one or more of the marine or other collagen type component(s) to cause the relevant marine or other collagen type component(s) to include greater than 70% tripeptides.

In some examples, one or more marine or other collagen type component(s) of the composition may include from about 71% to about 99% collagen, by weight. More specifically, one or more marine or other collagen type component(s) of the composition may include, for example, from about 72% to about 98% collagen, by weight. As a specific, nonlimiting example, one or more marine or other collagen type component(s) of the composition may include from about 75% to about 95% (e.g., about 77%, about 80%, about 82%, about 85%, about 88%, about 90%, about 92%) collagen, by weight.

To prepare compositions in accordance with this disclosure, such as essential oil food product formulations for animal or human consumption, at least one whole or partial CPTG essential oil component, at least one marine or other collagen type component having greater than 70% tripeptides, at least one whole or partial constituent component, and a liquid may be mixed until at least substantially homogenous. Following mixing, the composition may be prepared in a variety of material states and/or incorporated with other ingredients to form a final product. For example, the composition may be a spray dried powder, spray, oil, lipid, liquid, capsule, emulsion, dried powder, flakes, fast melt tablet, dissolvable film, liquid concentrate, effervescent tablet, softgel, gel, capsule, caplet, beadlet, or gum, one or more of which may be ingestible. As another example, the composition may be a cosmetic for topical use, such as, for example, a lotion, shampoo, conditioner, makeup, cream, soap, gel, foam, ear drops, eye drops, inhaler, mist, or ointment.

In some examples, one or more of the whole or partial CPTG essential oil component(s), the marine or other type collagen component(s), or the whole or partial constituent component(s) may be steam distilled, cold pressed, CO2 extracted, microwave extracted, solvent extracted, sublimated, or dehydrated before incorporation into the composition. As a specific, nonlimiting example, one or more whole or partial CPTG essential oils may be steam distilled from a root, rhizome, peel, stem, flower, seed, bark, or leaf at a rate of about 0.1 μg/day to 50,000 mg/day. Drying may involve, for example, rapid zone drying, window refractance, or vacuum belt drying.

A ratio by weight of essential oil collagen in the composition may be within a specified range. For example, a ratio by weight of the one or more essential oil component(s) to the one or more marine collagen component(s) may be from about 2:1 to about 10:1. More specifically, the ratio by weight of the one or more essential oil component(s) to the one or more marine collagen component(s) may be, for example, from about 3:1 to about 9:1. As a specific, nonlimiting example, the ratio by weight of the one or more essential oil component(s) to the one or more marine collagen component(s) may be from about 4:1 to about 8:1 (e.g., about 5:1, about 6:1, about 7:1).

The foregoing description of illustrative examples has been presented for purposes of illustration and of description. It is not intended to be exhaustive or limiting with respect to the precise form disclosed, and modifications and variations are possible in light of the above teachings or may be acquired from practice of the disclosed aspects of the present invention. It is intended that the scope of the present invention be defined by the claims appended hereto and their equivalents.

What is claimed is:

1. A composition, comprising:
at least one whole or partial CPTG essential oil component; and
at least one marine or other collagen type component comprising greater than 70% tripeptides; and
at least one whole or partial constituent component.

2. The composition of claim 1, comprising at least five whole or partial CPTG essential oil components, at least nine marine or other collagen type components, and at least twelve whole or partial constituent component.

3. The composition of claim 2, wherein at least four of the at least nine marine or other collagen type components comprise greater than 70% tripeptides.

4. The composition of claim 1, wherein the composition is a spray dried powder, spray, oil, lipid, liquid, capsule, emulsion, dried powder, flakes, fast melt tablet, dissolvable film, liquid concentrate, effervescent tablet, softgel, gel, capsule, caplet, beadlet, or gum.

5. The composition of claim 1, wherein the composition is ingestible.

6. The composition of claim 1, wherein the composition is a cosmetic for topical use, the cosmetic comprising a lotion, shampoo, conditioner, makeup, cream, soap, gel, foam, ear drops, eye drops, inhaler, mist, or ointment.

7. The composition of claim 1, further comprising at least one of extracts, spices, herbs, phytonutrients, vitamins, minerals, or food additives for preservation of taste, freshness, or mouthfeel, or a combination thereof.

8. The composition of claim 1, wherein the at least one marine or other collagen type component comprises from about 71% to about 99% collagen, by weight.

9. The composition of claim 1, wherein the composition is free of artificial flavors, preservatives, colors, binders, or fillers.

10. The composition of claim 1, wherein a ratio by weight of the at least one whole or partial CPTG essential oil component to the at least one marine or other collagen type component is from about 2:1 to about 10:1.

11. A method for preparing an essential oil food product formulation for animal or human consumption, comprising:
mixing at least one whole or partial CPTG essential oil component, at least one marine or other collagen type component comprising greater than 70% tripeptides, at least one whole or partial constituent component, and a liquid until at least substantially homogenous.

12. The method of claim 11, comprising steam distilling, cold pressing, CO₂ extracting, microwave extracting, solvent extracting, sublimating, or dehydrating at least one of the at least one whole or partial CPTG essential oil component, the at least one marine or other collagen, or the at least one whole or partial constituent component.

13. The method of claim 12, wherein drying comprises rapid zone drying, window refractance, or vacuum belt drying.

14. The method of claim 11, comprising steam distilling the at least one whole or partial CPTG essential oil from a root, rhizome, peel, stem, flower, seed, bark, or leaf at a rate of about 0.1 μg/day to 50,000 mg/day.

15. The method of claim 11, comprising performing enzymatic hydrolysis utilizing endo- and exo-peptidase enzymes on the at least one marine or other collagen type component to cause the at least one marine or other collagen type component to comprise greater than 70% tripeptides.

16. A composition comprising:
at least one essential oil component; and
at least one marine or other collagen type component,
wherein a ratio by weight of the at least one essential oil component to the at least one marine or other collagen type component is within a range of from about 2:0 to about 10:1.

17. The composition of claim 16, wherein the at least one marine or other collagen type component comprises at least nine types of collagen tripeptides.

18. The composition of claim 16, wherein the at least one essential oil component comprises at least five essential oil components, the at least five essential oil components comprising essential oil components derived from grapefruit peel, lemon peel, peppermint plant, ginger root, and cinnamon bark.

19. The composition of claim 16, further comprising one or more of nicotinamide mononucleotide (NMN) and resveratrol.

20. The composition of claim 16, wherein the at least one marine or other collagen type component comprises greater than 70% by weight tripeptides.
